(19) United States
(12) Patent Application Publication
(10) Pub. No.: US 2020/0392462 A1
(43) Pub. Date: Dec. 17, 2020

(54) METHODS OF FORMING CARDIOMYOCYES

(71) Applicants: THE REGENTS OF THE UNIVERSITY OF COLORADO, A BODY CORPORATE, Denver, CO (US); THE METHODIST HOSPITAL, Houston, TX (US)

(72) Inventors: Christopher J. Tsao, Houston, TX (US); Ennio Tasciotti, Latina (IT); Jeffrey Jacot, Boulder, CO (US); Francesca Taraballi, Houston, TX (US)

(21) Appl. No.: 16/964,548

(22) PCT Filed: Jan. 29, 2019
(86) PCT No.: PCT/US2019/015530

§ 371 Date: Jul. 23, 2020

Related U.S. Application Data
(60) Provisional application No. 62/623,074, filed on Jan. 29, 2018.

(51) Int. Cl. C12N 5/077; C08K 3/02; C12N 5/00; C12N 5/074

(52) U.S. Cl. CPC C12N 5/0657; C08K 3/02; C12N 5/0031; C12N 5/0607; B82Y 5/00; C08K 2003/023; C12N 2501/415; C12N 2506/45; C12Y 207/11026

(57) ABSTRACT

Methods of inducing cardiomyocytes from induced pluripotent stem cells by contacting induced pluripotent stem cells with silica nanoparticles comprising regulators of canonical Wnt signaling and coated with a biodegradable polymer.

### CROSS REFERENCE TO RELATED APPLICATION

This application claims the benefit of U.S. Provisional Patent Application Ser. No. 62/623,074, filed Jan. 29, 2018, which is incorporated herein by reference.

### STATEMENT OF FEDERALLY SPONSORED RESEARCH

This invention was made with government support under grant number CBET1547838 awarded by the National Science Foundation. The U.S. government has certain rights in the invention.

### TECHNICAL FIELD

This invention relates generally to the field of regenerative medicine and specifically to efficient methods of differentiating iPSC to functional cardiomyocytes.

### BACKGROUND

Cardiomyocytes(CM) are terminally differentiated cells which do not proliferate. Therefore, cardiac tissue damaged by disease or injury cannot repair itself, leading to arrhythmias and heart failure. Induced pluripotent stem cells (iPSC) represent a renewable and scalable cell source for cardiac tissue engineering applications and treatments. Unfortunately, current cardiac tissue engineering strategies are limited by a source of functional cardiomyocytes. Additionally, current procedures for differentiating iPSC into cardiomyocytes involve numerous manual media changes and inhibitor additions slowing cardiomyocyte formation and providing many opportunities for operator error and the introduction of contaminants, including microbial contamination. Thus, a method of inducing cardiomyocytes from stem cells while limiting handling interactions would be ideal for clinical translation of iPSC to cardiac cells.

As for the delivery, porous particles have been developed for pharmaceutical drug delivery due to their potential to control (delay) drug release, enhance drug dissolution, promote drug permeation across the intestinal cell wall (bioavailability) and improve drug stability under the extreme environment of the gastro-intestinal tract when administered orally (Vallhov H, Gabrielsson S, Strømme M, et al. Mesoporous silica particles induce size dependent effects on human dendritic cells. Nano Lett 2007; 7:3576-82; Fadeel B, Garcia-Bennett A E. Better safe than sorry: Understanding the toxicological properties of inorganic nanoparticles manufactured for biomedical applications. Adv Drug Deliv Rev 2010; 62:362-74).

### SUMMARY

The inventors have developed a method to temporally control the release of stem cell differentiation factors, such as GSK3/Wnt small molecule inhibitors, from porous silica nanoparticles to drive cardiac differentiation of iPSC without manual intervention. The porous silica particles are loaded with stem cell differentiation factors, coated with a biodegradable polymer, and contacted with iPSC. The contacted iPSC cells display both early and late cardiac markers, and begin spontaneous contraction at 3.0±0.6 Hz at 15-21 days after contact with the iPSC. This demonstrated the cardiac differentiation of pluripotent stem cells by porous silica vectors with temporally controlled release small molecules inhibitors. This method may be automated methods to reduce and eliminate variability in manual maintenance of inhibitor concentrations in the differentiation of pluripotent stem cells to cardiomyocytes.

Thus, in one aspect, this disclosure provides a method for inducing cardiomyocyte differentiation in induced pluripotent stem cells (iPS cells) by contacting iPS cells in in vitro cell culture media with a porous silica nanoparticle comprising a compound selected from the group consisting of a glycogen synthase kinase 3 (GSK3) inhibitor, a Wnt signaling inhibitor, and a combination thereof, to induce differentiation of cardiomyocytes. In these methods, the Wnt signaling inhibitor may be porcupine inhibitor IWP2. In these methods, the GSK-3 inhibitor may be CHIR99021. In these methods, the porous silica nanoparticle may comprise both the Wnt signaling inhibitor IWP2 and the GSK-3 inhibitor CHIR99021. In these methods, contacting the cells may include simultaneously contacting the iPS cells with a porous silica nanoparticle comprising the Wnt signaling inhibitor IWP2, and a porous silica nanoparticle comprising the GSK-3 inhibitor CHIR99021.

In these methods, the cell culture media may be a defined medium. In these methods, the cell culture media may be a serum free medium.

In these methods, the contacting of the silica nanoparticles and the iPSC may be conducted in a bioreactor. The bioreactor may comprise a vessel formed of a flexible or partially flexible, or rigid disposable container coupled to a means for mixing liquid contents in the disposable container; at least one media introduction port in fluid contact with the disposable container; at least one media removal port in fluid contact with the disposable container. The bioreactor may also be configured with retention screen(s) formed by pores in and opening through the disposable container. Such pores may be sized to retain the porous silica nanoparticles in the disposable container thereby allowing fluid cell culture media removal from the vessel without losing the cultured cells. In this way, the methods of inducing cardiomyocytes of this disclosure may be conducted in a bioreactor by introducing fluids through the at least one introduction port and removing fluid cell culture media through the at least one media removal port from the vessel to maintain a substantially steady-state equilibrium of fluid volume within the disposable container throughout the differentiation of cardiomyocytes.

Cardiomyocytes induced by the methods of this disclosure may thereafter be transplanted into a mammal in need of such treatment, such as a patient suffering from a heart disease.

The porous silica nanoparticles that are contacted with the iPSC may be coated with a biodegradable polymer to achieve delayed-release kinetics of the compound from the nanoparticle. In these methods, the polymer may be, for example, poly(dl-lactide-co-glycolide) (PLGA), photodegradable poly(ethylene glycol) (PEG), photodegradable poly(caprolactone) (PCL), photodegradable poly(L-lactide) (PLLA), or combinations of these polymers.

In these methods, the porous silica nanoparticles have an average particle size range between 2 and 20 micrometers in diameter. In these methods, the porous silica nanoparticles may have a particle shape comprising spheres or rod-shaped particles. In these methods, the porous silica nanoparticles may have an average zeta potential between −35 and −40.

Thus, another aspect of this disclosure is a porous silica nanoparticle comprising a compound selected from the group consisting of a glycogen synthase kinase 3 (GSK3) inhibitor, a Wnt signaling inhibitor, and a combination thereof. These silica nanoparticles may be coated with a biodegradable polymer to achieve delayed-release kinetics of the compound from the nanoparticle. The polymer may be, for example, poly(dl-lactide-co-glycolide) (PLGA), photodegradable poly(ethylene glycol) (PEG), photodegradable poly(caprolactone) (PCL), photodegradable poly(L-lactide) (PLLA), or combinations of these polymers. These silica nanoparticles may have an average particle size range between 2 and 20 micrometers in diameter. These silica nanoparticles may have a particle shape comprising spheres or rod-shaped particles. These silica nanoparticles may have an average zeta potential between −35 and −40.

This Summary is neither intended nor should it be construed as being representative of the full extent and scope of the present disclosure. Moreover, references made herein to “the present disclosure,” or aspects thereof, should be understood to mean certain embodiments of the present disclosure and should not necessarily be construed as limiting all embodiments to a particular description. The present disclosure is set forth in various levels of detail in this Summary as well as in the attached drawings and the Description of Embodiments and no limitation as to the scope of the present disclosure is intended by either the inclusion or non-inclusion of elements, components, etc. in this Summary. Additional aspects of the present disclosure will become more readily apparent from the Description of Embodiments, particularly when taken together with the drawings.

### BRIEF DESCRIPTION OF THE FIGURES

FIG. 1A shows a schematic showing formulation of CHIR99021 and IWP2 loaded pSi. Gray areas represent porous silica and light gray area represents PLGA coatings. FIG. 1B is a TEM image of a single porous silica particle (scale 20 nm). FIG. 10 is a SEM image of pSi (scale 2 μm). FIG. 1D is a SEM image of PLGA-pSi (scale 10 μm). FIG. 1E is a confocal image of FITC-labeled pSi within PLGA coating (scale 10 μm). FIG. 1F is a table of Zeta potential and particle diameters. FIG. 1G is a graph showing the cumulative release profiles of CHIR99021 and IWP2. FIG. 1H shows small molecule loading efficiencies into pSi and PLGA-pSi (n=3).

FIG. 2A shows the internalization of pSi and PLGA-pSi in AFSC-iPSC after 72 hours.

FIG. 2B shows the apoptosis and necrosis of AFSC-iPSC after 72 hours (n=3).

FIG. 3A is a schematic showing transwell differentiation with pSi encapsulated inhibitors. FIG. 3B shows flow cytometry results showing cardiac differentiation efficiency represented by cTnT expression at day 12 (n=3, p<1.05 is significant, *significant vs other groups). FIG. 3C shows Live/Dead and bright field images of differentiating cells.

FIG. 4A is a timeline of pSi differentiation and cardiac marker analysis. FIG. 4B shows Brachyury expression compared to D(−3). FIG. 4C shows Oct 4 expression compared to D(−3). FIGS. 4D and 4E show early markers of cardiac differentiation (Nkx2.5, 151-1) compared to DO. FIGS. 4F, 4G, and 4H show late markers of cardiac differentiation (cTnT, β-MHC, Cx43) compared to DO. In these figures, n=3, p<1.05 is significant, * indicates significant difference from day 0, # significant difference from day −3.

FIG. 5 shows confocal immunofluorescence of differentiated AFSC-iPSC. Top series of images are cells differentiated with pSi particles. Bottom series of images are cells differentiated in 2D with dissolved inhibitors. Scale 20 μm.

FIG. 6A shows calcium (Indo-1) Voltage (Di-8-ANEPPS) sensitive dye traces for pSi and 2D differentiation at day 30. FIG. 6B is a Western blot analysis of MYH7 and cTnT at day 30 (n=3). FIG. 6C shows averaged peak length shortening percentages at day 30 (n=3).

FIG. 6D shows averaged spontaneous beating frequency using transient dye measurement data. FIG. 6E is an image of beating cardiac cells within transwell (scale 20 μm).

### DETAILED DESCRIPTION

This disclosure provides methods for forming cardiomyocytes using a mesoporous silica delivery system that controls release and delivery of growth factors to induce differentiation of stem cells in vitro. These methods provide in vitro protocols for controlled and reproducible stem cell differentiation that can be translated to clinical application. The inventors' research has also led to the creation of a bioreactor, which provides a reproducible, controlled in vitro environment favorable to stem cell survival and differentiation, has great potential for use in developmental biology and stem cell transplantation treatments.

The methods of this disclosure may be directed to the production of cardiomyocytes for patients suffering cardiovascular disease, or patients requiring organs and tissues to be reinnervated after transplantation (e.g., cardiac transplants), or newly created organs/tissues from stem/progenitor cells of different sources. The method comprises using porous silica for the delivery of differentiation factors in the controlled and reproducible production of cardiomyocytes.

This disclosure also provides a pharmaceutically active ingredient for inducing stem cell differentiation to cardiomyocytes, which comprises porous silica containing releasable agents conducive for stem cell differentiation ex vivo.

The porous silica forming the nanoparticles used in these methods may be characterized by a surface area higher than 200 m²/g and a pore size between 1.5-50 nm. These porous silica particles can have a particle shape of spheres or rod-shaped particles. Preferably these particles are substantially spherical particles. Such porous silica particles are available for purchase from numerous commercial vendors. After loading one or more compounds effective to induce differentiation of stem cells into cardiomyocytes, the nanoparticles are coated with a biodegradable polymer, which functions to delay the release of the compound(s) from the nanoparticle into the in vitro cell culture containing the stem cells. Preferably, the biodegradable polymer is poly(dl-lactide-co-glycolide) (PLGA). These coated particles may have an average particle size and/or sizes ranging between 2 and 20 micrometers in diameter.

The releasable compound capable of contributing to stem cell differentiation into cardiomyocytes is preferably 1-60% of the total weight of the silica nanoparticle, and more preferably between 10-45 wt %.

The releasable compounds(s) loaded into the porous silica nanoparticles are selected from secreted growth factors and morphogens, including, but not limited to fibroblast growth factors (FGFs), Wnts protein family members, transforming growth factor (TGF)beta family members, Hedgehog (hh) proteins, retinoic acid, vascular endothelial growth factor (VEGF), Dickkopf (Dkk)-1, insulin, Activin, SDF-1/CXCL12), pleiotrophin (PTN), insulin-like growth factor 2 (IGF2), ephrin B1 (EFNB1), cAMP, regulators of canonical Wnt signaling such as IWP2, and Gsk3 inhibitors such as CHIR99021.

The stem cells that are contacted with the porous silica nanoparticles in the methods of this disclosure are preferably chosen from the group consisting of regional stem cells, embryonic stem (ES) cells, neural crest stem cells, neural stem cells from brain and spinal cord, mesenchymal stem cells, endothelial stem cells, endodermal stem cells, induced pluripotent stem (iPS) cells.

Cardiomyocytes have been generated in vitro from a wide range of stem/progenitor cells, including iPSCs (Gai H, et al. Generation and characterization of functional cardiomyocytes using induced pluripotent stem cells derived from human fibroblasts. Cell Biol Int. 2009; 33:1184-93; Kuzmenkin A, et al. Functional characterization of cardiomyocytes derived from murine induced pluripotent stem cells in vitro. FASEB J 2009; 23:4168-80; Pfannkuche K, et al. Cardiac myocytes derived from murine reprogrammed fibroblasts: intact hormonal regulation, cardiac ion channel expression and development of contractility. Cell Physiol Biochem 2009; 24:73-86), ESCs (Beqqali A, et al. Human stem cells as a model for cardiac differentiation and disease Cell Mol Life Sci 2009; 66:800-13; Steel D, et al., Cardiomyocytes derived from human embryonic stem cells-characteristics and utility for drug discovery. Curr Opin Drug Discov Dev 2009; 12:133-40), hematopoietic progenitor/stem cells, MSCs (Choi S C, et al., Specific monitoring of cardiomyogenic and endothelial differentiation by dual promoter-driven reporter systems in bone marrow mesenchymal stem cells. Biotechnol Lett 2008; 30:835-43; Antonitsis P, et al. Cardiomyogenic potential of human adult bone marrow mesenchymal stem cells in vitro. Thorac Cardiovasc Surg 2008; 56:77-82; Ge D, et al. Chemical and physical stimuli induce cardiomyocyte differentiation from stem cells. Biochem Biophys Res Commun 2009; 381:317-21; Gwak S J, et al. In vitro cardiomyogenic differentiation of adipose-derived stromal cells using transforming growth factor-beta. Cell Biochem Funct 2009; 27:148-54), and cardiomyocyte progenitor cells (Smits A M, et al. Human cardiomyocyte progenitor cells differentiate into functional mature cardiomyocytes: an in vitro model for studying human cardiac physiology and pathophysiology. Nat Protoc 2009; 4:232-43).

Intravascular delivery or cardiac transplants of multipotent or pre-differentiated cardiogenic cells from these stem cell sources have been shown to promote cardiac structural repair and functional restoration in animal models of myocardial injury (Fukushima S, et al. Choice of cell-delivery route for skeletal myoblast transplantation for treating post-infarction chronic heart failure in rat. PLoS One 2008; 3:e3071; Hendry S L 2nd, et al. Multimodal evaluation of in vivo magnetic resonance imaging of myocardial restoration by mouse embryonic stem cells. J Thorac Cardiovasc Surg 2008; 136:1028-37; Matsuura K, et al. Transplantation of cardiac progenitor cells ameliorates cardiac dysfunction after myocardial infarction in mice. J Clin Invest 2009; 119:2204-17; Jin J, et al. Transplantation of mesenchymal stem cells within a poly(lactide-co-epsilon-caprolactone) scaffold improves cardiac function in a rat myocardial infarction model. Eur J Heart Fail 2009; 11:147-53; Okura H, Matsuyama A, Lee C M, et al. Cardiomyoblast-like cells differentiated from human adipose tissue-derived mesenchymal stem cells improve left ventricular dysfunction and survival in a rat myocardial infarction model. Tissue Eng Part C Methods 2010; 16:417-25).

The encouraging results from this experimental research have prompted several clinical trials in patients with myocardial disease, using different types of progenitor/stem cells (Segers V F, Lee R T. Stem-cell therapy for cardiac disease. Nature 2008; 451:937-42; Joggerst S J, Hatzopoulos A K Stem cell therapy for cardiac repair: benefits and barriers. Exp Rev Molec Med Epub 2009 Jul. 8; 11:e20; Piepoli M F. Transplantation of progenitor cells and regeneration of damaged myocardium: more facts or doubts? Insights from experimental and clinical studies. J Cardiovasc Med 2009; 10:624-34).

Cardiomyocyte progenitors were generated ex vivo from stem cells contacted with inhibitors of the GSK3/Wnt signaling pathways. The release of these inhibitors was temporally controlled by first loading one or both of these compounds into the porous silica nanoparticles described above, which were then coated with 5 wt % PLGA 50:50. The release of these inhibitors the porous silica nanoparticles to contact the stem cells in vitro cell culture can delay release of the compounds from the nanoparticles to the cell culture by about 72 hours.

Any of these methods described above may be configured, in any combination, within a disposable bioreactor vessel. The loaded and coated porous silica nanoparticles are placed in a storage space that is in fluid communication with a bioreactor vessel chamber. In the context of this disclosure, a first and a second location are in “fluid communication” if there is a structure between the two locations defining, e.g., a conduit, channel, passage, opening, etc., that facilitates fluid flow from the first location to the second location. For example, the silica nanoparticles may be placed in a vessel that is connected to a bioreactor vessel chamber by a media introduction line, or the silica nanoparticles may be placed directly into the media introduction line itself.

Sterile fluid media, which may include the silica nanoparticles, is added to the bioreactor vessel chamber. The fluid medium may include cell culture medium or nutrient solutions, or a combination of the two, as well as other fluids. Repeated additions of media fluid and/or silica nanoparticles can be made through the media introduction port to the bioreactor vessel chamber in which stem cells are sustained, grown, and/or differentiated into cardiomyocytes.

After the stem cells have grown to a sufficient density within a bioreactor vessel, it may be desirable to remove the spent cell culture medium from the bioreactor for a variety of reasons, including performing an exchange of spent medium with fresh medium. In such cases, it is desirable to remove only the spent medium from the bioreactor vessel while retaining the stem cells within the bioreactor vessel chamber. The fluid may be removed from the bioreactor vessel through media removal port in fluid contact with the container that forms the bioreactor vessel. The media removal port may contain one or more screens or filters that allow removal of fluid without removing cardiomyocytes or stem cells from the bioreactor vessel.

Additional gases may be injected into the bioreactor vessel through a sparger. A harvest port allows for the final removal of the cardiomyocytes from the bioreactor vessel. Means to mix or agitate the fluid in the bioreactor vessel may be used within the bioreactor. Fluid(s) are preferably introduced through the at least one introduction port and removed through the at least one removal port to maintain a substantially steady-state equilibrium of fluid volume within the container bioreactor vessel throughout the process of differentiation of cardiomyocytes.

### EXAMPLES

The following methods were used to conduct experiments described in Examples 1-6, below:

Cells source: Cells were isolated from second-trimester human amniotic fluid and reprogrammed to induced pluripotent stem cells (iPSC) through mRNA transfection as previously reported (Gao, Y., et al. Annals of biomedical engineering 42, 2490, 2014). Pluripotency was assessed through RNA expression of pluripotency markers OCT4, nanog, and Tra-1-81 with teratoma formation in a nude mouse as previously reported (Velasquez-Mao, A., PLoS One 12 (5), e0177824, 2017). Reprogrammed AFSC were then passaged onto a mouse embryonic fibroblast feeder layer (1.85×10⁴ cells/cm²) for further expansion and maintained in knockout serum replacement induced pluripotent stem cell media (DMEM/F12, 20% knockout serum replacement, 1% NEAA, 1% Pen/Strep, 0.1% beta-mercaptoethanol, 4 ng/ml bFGF). At 60-70% confluency, colonies were passaged to new mouse embryonic fibroblast feeder layers, every 7-10 days.

Porous silica particle synthesis: A tannic acid template and the Stöber process were used to synthesis pSi as per previous studies (Parodi, A., et al., ACS nano 8, 9874, 2014). Briefly, 272 mg Tannic acid was dissolved in 50 ml ethanol while continuously stirring. Then 25 ml ammonium hydroxide was added and stirred for 1 min. Tetraethyl orthosilicate (300 μl) was added to the mixture dropwise and stirred continuously for 3 hours. The resulting particles were centrifuged and washed in 1:1 ethanol:water for a total of 6 washings. The particles were then resuspended in 15 ml of deionized water and filtered twice through 40 μm centrifuge tube filters. Particles were centrifuged again, the water was removed and then resuspended in 1 ml isopropyl alcohol. The particles were then dried overnight in a vacuum oven at 60° C. and −36 Pa until dry. Once dry, 4 mg of pSi were rehydrated in 0.5 ml inhibitor loading solutions, consisting of CHI R99021 or IWP2 re-suspended in DMSO at 600 ug/ml for 20 min at 37° C. with constant mixing. The particles were then washed and lyophilized for further use.

PLGA encapsulation of porous silica particles: Particles loaded with IWP2 were further encapsulated with a PLGA coating to delay the release kinetics using a Solid-in-Oil-in-Water method (S/O/VV). An oil solution consisting of dissolved 5 wt % 50:50 PLGA in dichloromethane was homogenized with the prepared loaded pSi. The solid in oil emulsion was then added dropwise to a water solution consisting of 2.5% polyvinyl alcohol. The solution was stirred continuously for 6 hours, washed and lyophilized until ready for use.

Particle characterization: Dynamic light scattering (DLS) and Z-potential of pSi were performed using a Zetasizer ZEN3600 (Malvern, Worcestershire, U.K.). Moreover, samples were prepared for Scanning electron microscope (SEM) by drying overnight on a stage and sputter coating with a 5 nm thick layer of Pt/PI. SEM were taken using Nova NanoSEM 230. PLGA-pSi and pSi size distribution were also measured with the software ImageJ (NIH Image). TEM samples were prepared by drying nanoparticles onto 300 mesh carbon-coated copper grids and then analyzed. FITC-labeled pSi has been used to verify the presence of the silica inside the PLGA shell by confocal microscopy.

Inhibitor loading efficiency and release studies: All supernatants after initial loading of inhibitors were stored at −20° C. Supernatants were analyzed by reverse phase high performance liquid chromatography (HPLC). Inhibitor release studies were done sampling particle suspension inhibitor release up to 6 days. Briefly, drug release solution was prepared using 1 mL of 1×PBS, and 1 mL of 1% bovine serum albumin. The lyophilized pSi were suspended at a concentration of 3 mg/mL of drug release solution and incubated at 37° C. with constant stirring. At 1, 3, 5, 12 and every 24 hours thereafter, suspensions were centrifuged and 1 ml drug release solution was taken and replaced with fresh drug release solution. Agilent Zorbax Eclipse Plus C18 (100×4.6 mm, 3.5 um pore particle size) column was used for analysis. Samples flowed through the column at 1.0 ml/min and with a mobile phase consisting of acetonitrile in 0.1% trifluoroacedic acid: H₂O in 0.1% trifluoroacedic acid (V/V). Samples were analyzed at 281 nm.

Cellular toxicity to particles: pSi without any inhibitors loaded were added to iPSC cultures at a density of 2.5 ng/cell to assess apoptosis and necrosis. Annexin V and propidium iodide staining were used to quantify apoptosis/necrosis. Cells were lifted at 0 and 72 hours, washed and double stained with Annexin V-FITC and propidium iodide. The cells were then analyzed through flow cytometry. To access the effect of transwell culture on cell survival, live/dead staining was done according to manufacturer protocol (Thermo Fisher). Cells were imaged using fluorescent microscopy.

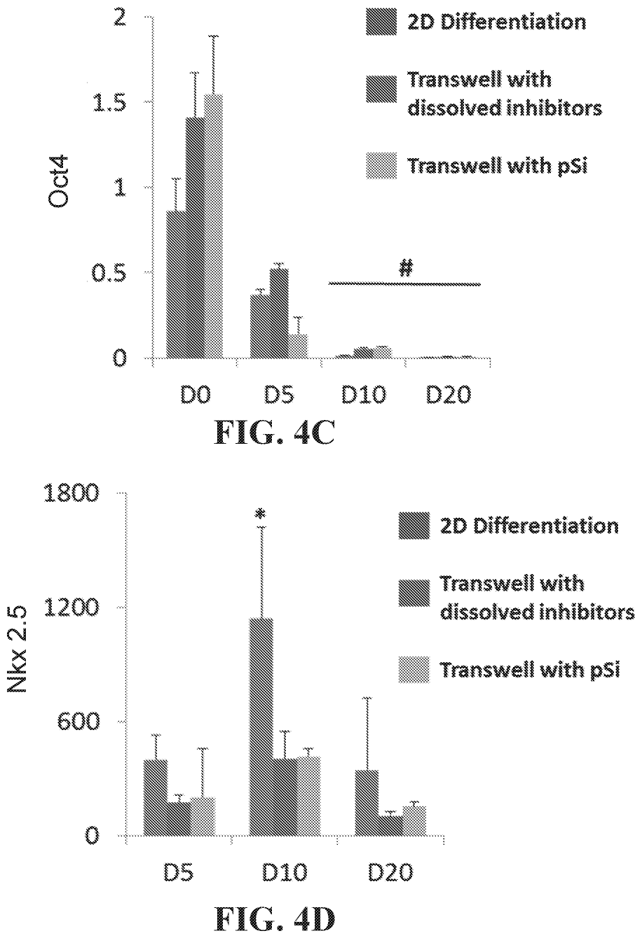

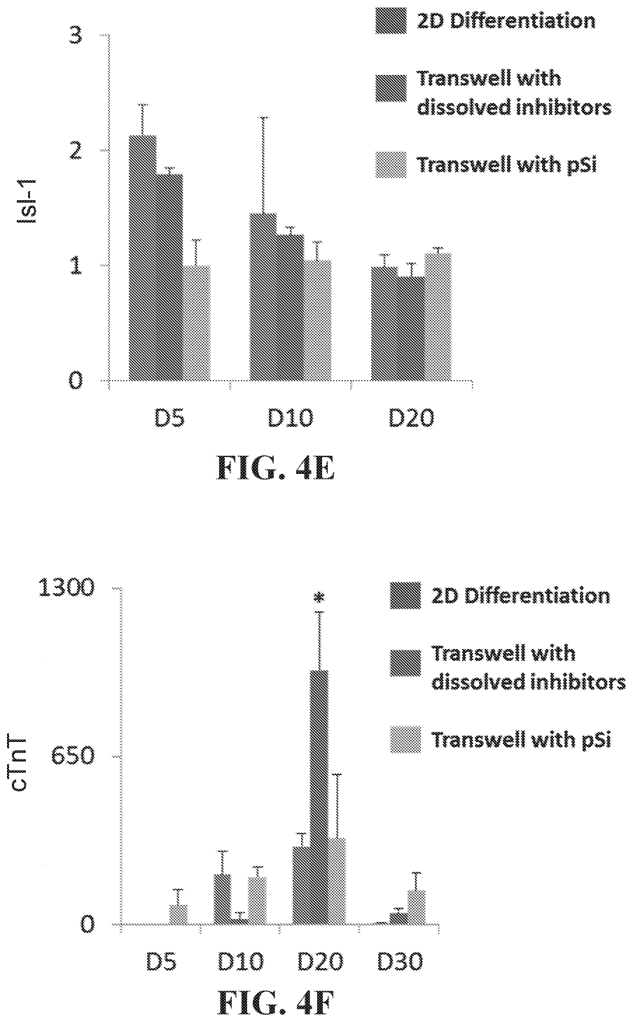

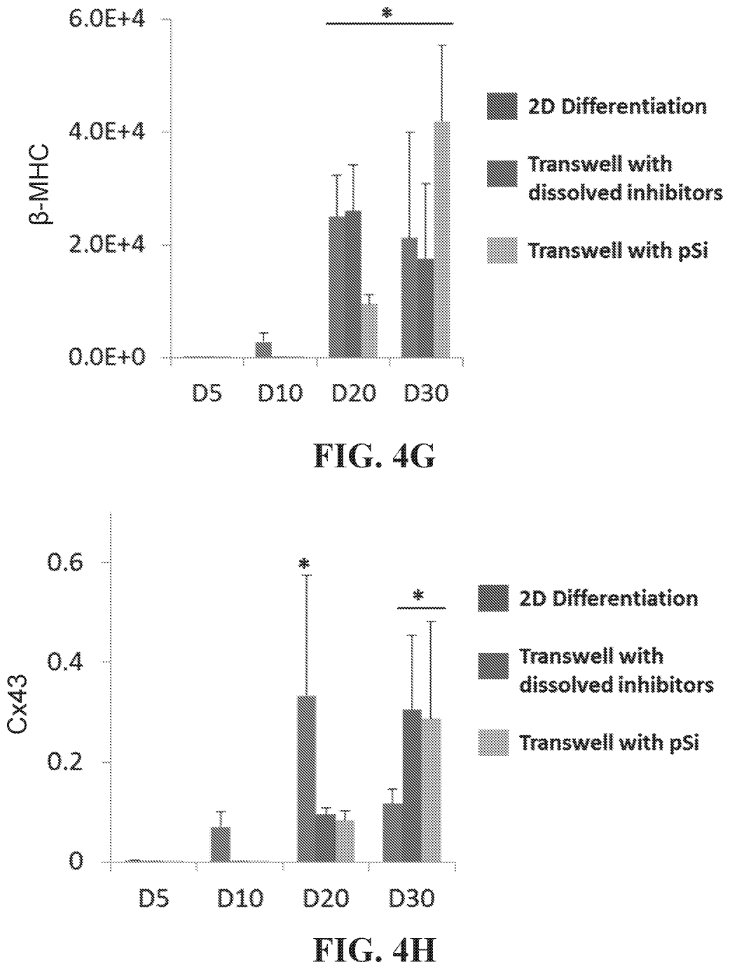

Differentiation of iPSC using dissolved GSK3/Wnt Inhibitors: By adapting previously published protocols (Lian, X., et al., Nature protocols 8, 162, 2013), AFSC-iPSC were differentiated into cardiac cells by temporally inhibiting the GSK3 and Wnt signaling pathways over a span of 5-7 days (experimental timeline shown in FIG. 4A). Upon reaching approximately 70% confluency, undifferentiated colonies were dissociated in collagenase type 2 for 5 min then manually dislocated from the feeder layer, dispersed into single cell suspension, then plated as a monolayer of cells onto MATRIGEL™ at 260,000 cells/cm². Cells were expanded 3 days in mTeSR1 media (STEMCELL Technologies, Cambridge, Mass.). Media was then changed to RPMI 1640 and the GSK3 inhibitor, CHIR99021, was added at a concentration of 6 μM, representing differentiation Day 0. After 24 hours, media was replaced with fresh RPMI 1640. At day 3 the Wnt inhibitor, IWP2, was added to RPMI/B27 without insulin at a concentration of 2.5 μM. At day 7, insulin and ascorbic acid were added to the RPMI 1640 media. The occurrence of beating colonies was monitored through phase contrast microscopy after day 7.

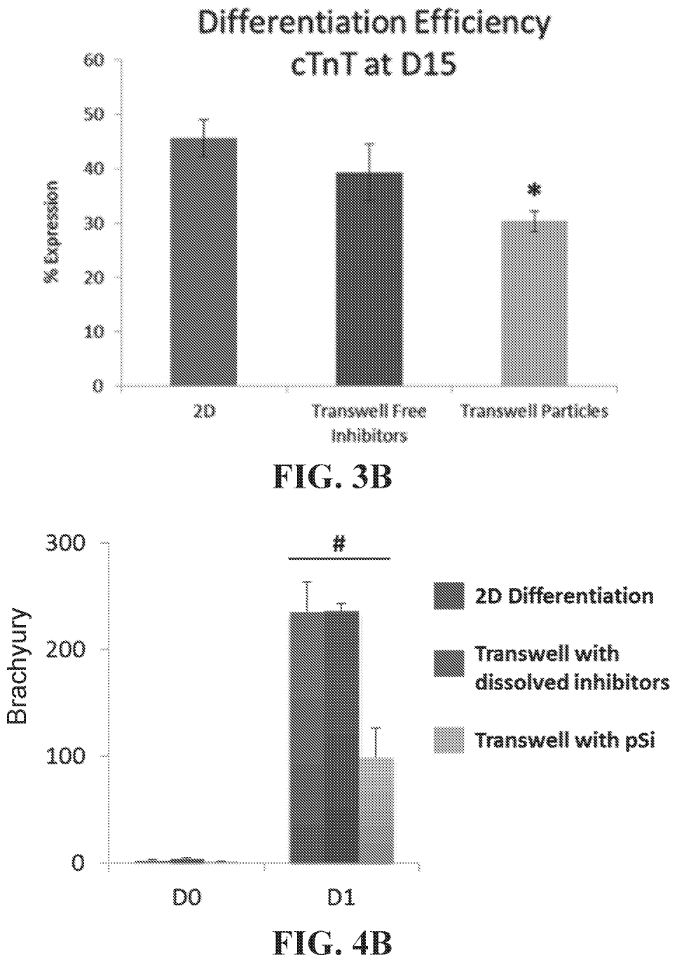

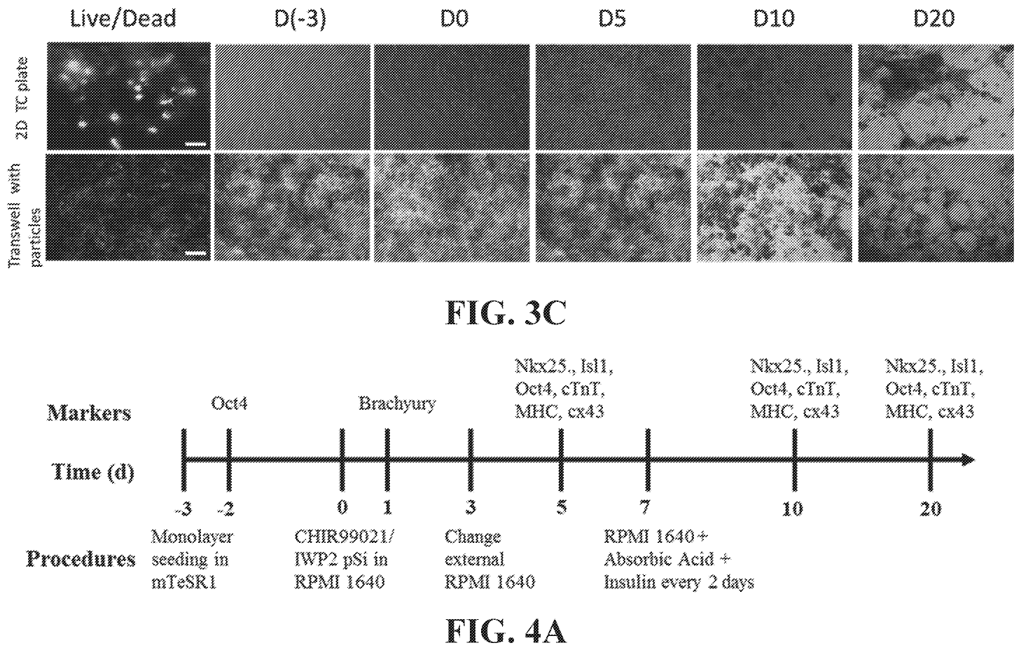

Differentiation of iPSC using pSi-released inhibitors: To test the effectiveness of inhibitor release from loaded pSi, dissociated iPSC were plated at 260,000 cells/cm² onto MATRIGEL™ coated 12-well polyethylene terephthalate ThinCert cell culture inserts with 0.4 μm pore size (schematic of transwell shown in FIG. 3A). Based on calculated loading efficiencies, approximately 1.5 mg CHIR99021 loaded pSi and 4.0 mg IWP2 loaded PLGA-pSi were suspended in 24 ml of RPMI 1640. At day 0 of differentiation, 1 ml of particle suspension was added to each seeded transwell and an additional 1 ml RPMI 1640 surrounded the insert. Every 48 hours, 1 ml of fresh RPMI1640 replaced the media surrounding the insert. After day 8, insulin and ascorbic acid were added to the RPMI 1640 and 1 ml of media was added each to the insert and surrounding.

Flow Cytometry: Cells were detached at day 12 into suspension with ACCUTASE™ and stained with a fluorescently conjugated antibody for cardiac troponin T at a dilution of 1:100. BD Acurri C6 Plus software was used for all flow cytometry data collection. FLOWJO™ software was used for data analysis.

Gene Expression Analysis: Gene expression was quantified using qRT-PCR. To assess the upregulation of early and late stage cardiac markers, cell samples were collected at 0, 1, 5, 10, and 20 days after the start of differentiation. At each time point, RNA samples were collected using an RNA collection kit following manufacturer protocol. mRNA samples were then reverse transcribed to DNA using a cDNA kit following manufacture protocol. The resulting DNA samples were analyzed with quantitative real-time polymerase chain reaction (qRT-PCR) using a proprietary assay system following manufacturer protocol. All samples were assayed for expression of Oct4, Isl1 Nkx2.5, cTnT, connexin 43, myosin heavy chain, and GAPDH as a house keeping gene using DNA primers. The expression of each gene was first normalized to the level of GAPDH. Relative fold changes were determined by calculating ΔΔCt comparing to Day (−3) or Day 0. Biological triplicates for each group were assessed, and results were reported as mean±standard deviation.

Immunofluorescence: Cell cultures were fixed in 4% paraformaldehyde at 4° C. for 20 minutes. Fixed cells were permeated with Triton X100 for 5 min at room temperature. Next, cells were incubated with specific antibodies for cardiac markers myosin heavy chain and connexin 43, then in DyLight-conjugated secondary antibodies. Cells were imaged using an epifluorescence microscope.

Calcium handling and membrane voltage potential: The calcium handling of spontaneously contracting cells was measured by imaging calcium-sensitive dye Indo-1 AM with an epifluorescence microscope and photomultiplier tube detection system and software. Cells were washed with PBS at 37° C., replaced with of Tyrode's buffer containing 2 μl of 2 mM Indo-1 AM, and allowed to incubate 30 min at room temperature. The cells were then washed three times with fresh Tyrode's buffer and analyzed using the Ion Optix system. The measured field of view was approximately 50 μm×50 μm in size. Detection wavelengths were recorded at 405 nm and 485 nm. Membrane voltage potential utilized the same procedure as calcium measurements but with voltage sensitive dye Di-8-ANEPPS. Detection wavelengths were recorded at 560 nm and 620 nm.

Peak shortening percentage analysis: Cells were recorded upon spontaneous contraction from day 20-30 using phase contrast time-lapse video microscopy (Nikon Eclipse TE300). Images were taken every 100 ms with a CoolSNAP HQ2 CCD camera and analyzed with ImageJ. Minimum representative rectangles were used to outline individual contracting cells and the lengths of the smallest and largest rectangles were used to calculate the peak shortening percentage.

Western Blot: Western blot antibodies were purchased from Abcam Inc.; electrophoresis, transfer blots, and ECL developing materials were purchased from Bio-Rad. Total protein lysates were collected from cells at 30 days of differentiation. Total protein concentration was quantified by a bicinchoninic acid kit (BCA; Thermo Scientific). Samples were normalized based on total protein concentration and run on SDS-PAGE gels according to the manufacturer's protocols. Proteins were transferred to blotting membranes and then incubated overnight at 4° C. with mouse monoclonal antibodies against MHC and cTnT (1:100 dilution in 10% milk in Tris Buffered Saline with 0.5% Tween 20 (TBST)) and mouse monoclonal antibodies against GAPDH (1:1000 dilution in 10% milk in TBST). The membranes were washed and incubated with secondary HRP antibodies (1:1000 in 10% milk in TBST) for 1 hour at room temperature. The membranes were washed, immersed in a ECL developing solution, and then read using a Bio-rad ChemiDoc XRS+. Statistics.

Statistics: Statistical analyses were done in SigmaPlot. Data were compared using one-way analysis of variance (ANOVA) followed by Tukey's test, p<0.05 was considered significant. Results were presented as mean±standard deviation with number of samples/trials indicated in the brief description of the Figures.

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

The following are examples of methods and compositions of the invention. It is understood that various other embodiments may be practiced, given the general description provided above.

### Example 1

### pSi Characterization and GSK3 and Wnt Inhibitor Release from pSi

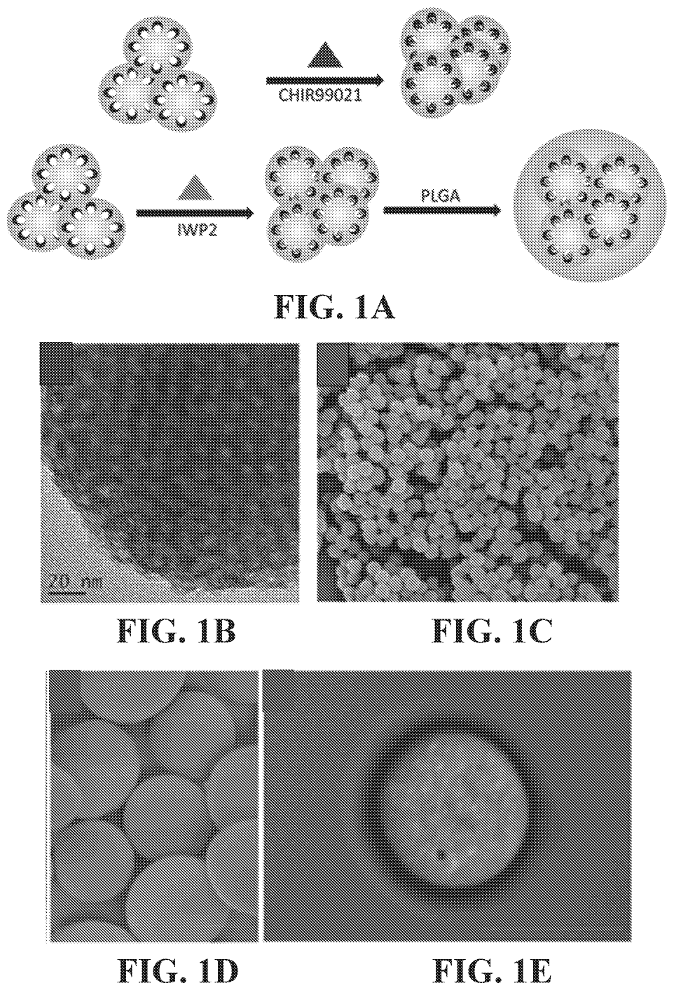

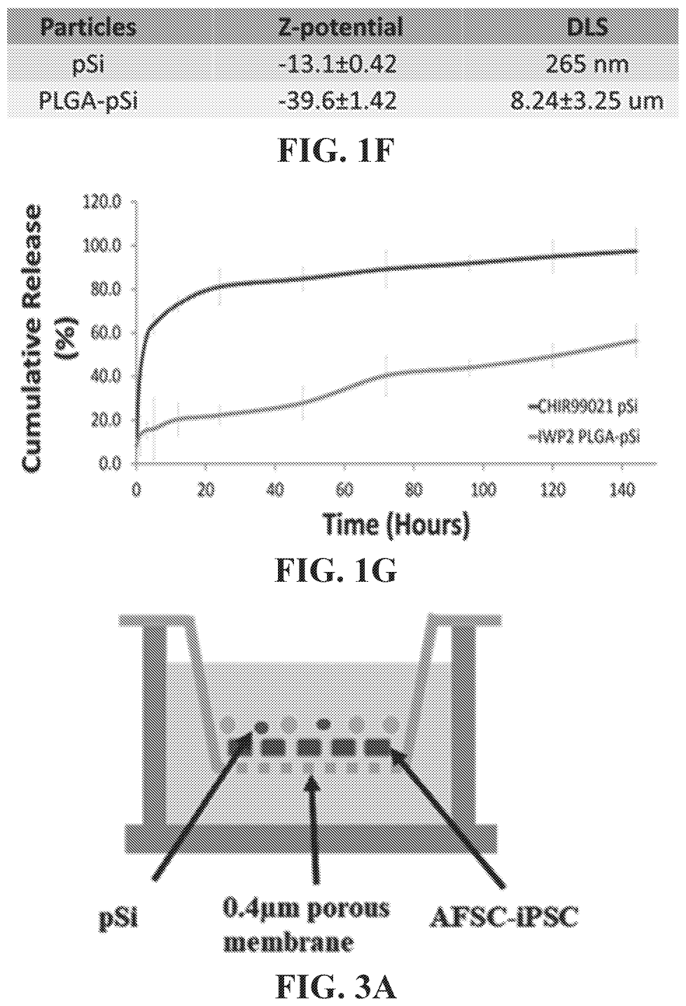

The inventors created PLGA-pSi particles that measured 8.24±3.25 μm in diameter and uncoated pSi particles that measured 265 nm in diameter, per dynamic light scattering (FIGS. 1A-1E). Zeta potentials for pSi and PLGA-pSi were −13.1±0.42 and −39.6±1.42, respectively (FIG. 1F). CHIR99021 and IWP2 were loaded into silica particles by direct absorption from a 600 μg/ml solution (FIG. 1H). HPLC data showed a burst release profile with 81.1±5.3% of CHIR99021 released within the first 24 hours (FIG. 1G). After the initial 24 hours, CHIR99021 continued to be released through day 7 with nearly 100% cumulative release at 144 hours (FIG. 1G).

When IWP2 was encapsulated within the porous silica particles and coated with 5 wt % PLGA 50:50, the results showed a cumulative release of 27.8±7.2% within the first 48 hours (FIG. 1G). The particles were shown to have 40.2±9.1% cumulative release at the end of 72 hours. After the 144 hours, IWP2 was shown to have a cumulative release of 56.4±7.2%.

### Example 2

### Cellular Toxicity with pSi Exposure

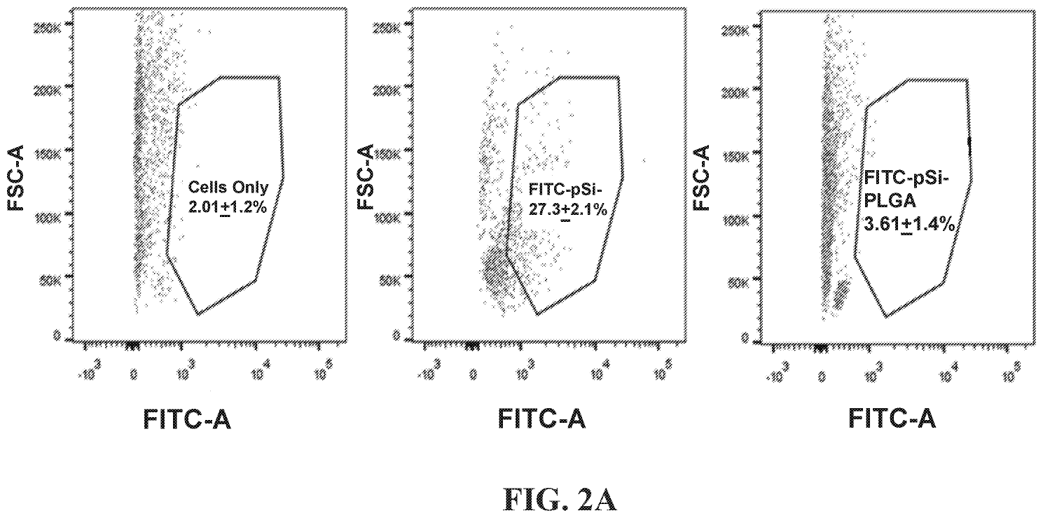

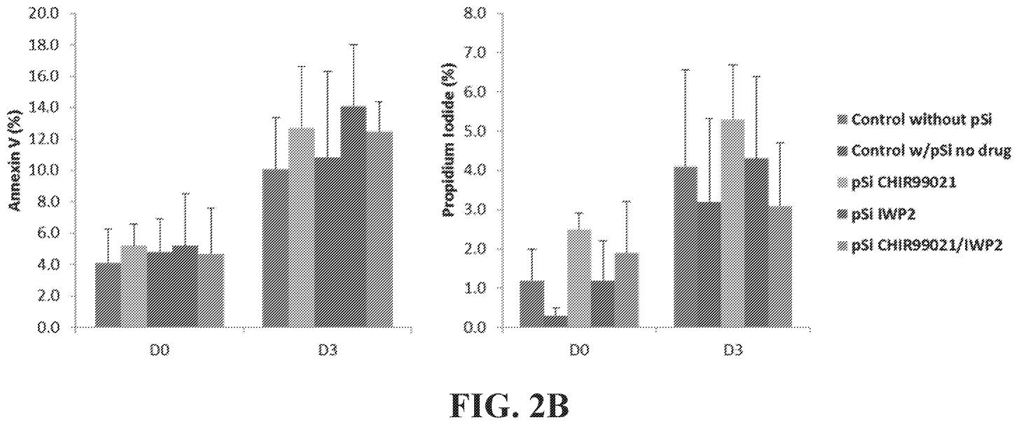

Forward scattering of annexin V labeled cells measured by flow cytometry showed that 27.3±2.1% of cells internalized the uncoated pSi and 3.61±1.4% of cells internalized PLGA-pSi after 3 days in culture (FIG. 2A). Uncoated pSi and PLGA-coated pSi were then tested for cellular toxicity through an annexin V and propidium iodide assay (FIG. 2B). After 3 days, annexin V staining of iPSC exposed to pSi loaded with both GSK3 and Wnt inhibitors (12.5±1.9%) showed no significant difference compared to the cells alone (10.1±3.3%) (FIG. 2B, left panel), suggesting no increase in apoptosis. Propidium iodide staining also showed no significant difference between iPSC exposed to pSi loaded with both inhibitors (3.1±1.6%) compared to cells alone (4.1±2.5%) (FIG. 2B, right panel), suggesting no increase in cellular necrosis.

### Example 3

### Cardiac Gene Expression in pSi Differentiation is Comparable to Dissolved Inhibitors

The inventors compared differentiation of iPSC cells in 2D monoculture and transwells (transwell schematic depicted in FIG. 3A). Neither the 2D monolayer culture nor the transwell culture showed any significant difference in viability after 3 days in culture (FIG. 3C). The morphology of differentiating cells in transwells at day 0 showed denser cell localization and less cell spreading compared to 2D monolayer differentiation. During the first 5 days of differentiation, the 2D monolayer differentiation had a phase-dark layer of fibroblastic cells below a layer of spherical phase-bright cells, while a phase-bright cell layer was absent in transwell culture. At time points greater than 10 days, both groups exhibited a clustered morphology with elongated web-like sheets (FIG. 3C). Spontaneous beating occurred in all groups at approximately 18-25 days of differentiation. Localized beating colonies were observed in all groups and were not synchronously paced.

cTnT expression showed 30.3±1.9% differentiation efficiency in pSi differentiated cells at day 15. Differentiation efficiency was greater in both 2D and transwell-dissolved inhibitor groups with 45.6±3.5% and 39.3±5.3%, respectively (FIG. 3B).

### Example 4

### pSi Released Inhibitor Induces Expressed Differentiation Markers of Cardiac Differentiation

Cardiac differentiation was monitored through gene expression of brachyury, early stage cardiac markers (nkx2.5 and isl-1), and later stage cardiac markers (cTnT, β-MHC, Cx43). Pluripotent stem cell marker OCT4 was also monitored throughout the differentiation to quantify remaining pluripotency in the cell population (FIGS. 4A-4H).

Brachyury expression was measured 24 hours after the introduction of CHIR99021 in both conditions. The relative expression compared to day (−3) of differentiation showed that all groups had little to no brachyury expression at the start of differentiation, but after 24 hours, all groups showed significant upregulation. The 2D monolayer differentiation and transwell dissolved inhibitors had a fold change increase of 236.3±6.7, where the transwell with inhibitor loaded particles showed a significant fold change increase of 99.3±27.1 (FIG. 4B).

When compared to day (−3), OCT4 expression was shown to be downregulated over the course of differentiation in all groups (FIG. 4C). At day 10 and 20, OCT4 fold expression in all conditions was below 0.06±0.01 and 0.007±0.006, respectively.

Early stage cardiac markers, Nkx2.5 and Isl-1, were both upregulated throughout the differentiation compared to day 0 (FIGS. 4D and 4E). At each of the time points, Nkx2.5 expression was lower in the transwell groups compared to the 2D monolayer group. When compared to the other time points within groups, day 10 showed significant upregulation of Nkx2.5 (FIG. 4D).

Cardiac troponin T (cTnT) was upregulated as early as day 5 in the transwell group with free inhibitors when compared to day 0 (FIG. 4F). However significant upregulation of cTnT was only shown in the pSi group at day 20 of differentiation. β-MHC is upregulated at day 10 in the 2D monolayer group, where later time points showed β-MHC significantly upregulated across all groups (FIG. 4G). At day 30, the transwell group with pSi had the highest expression of β-MHC. Gap junction protein connexin 43 (Cx43) expression was not significantly different compared to day 0 at each time point in all groups (FIG. 4H).

### Example 5

### Early Cardiac Maturation and Arrangement

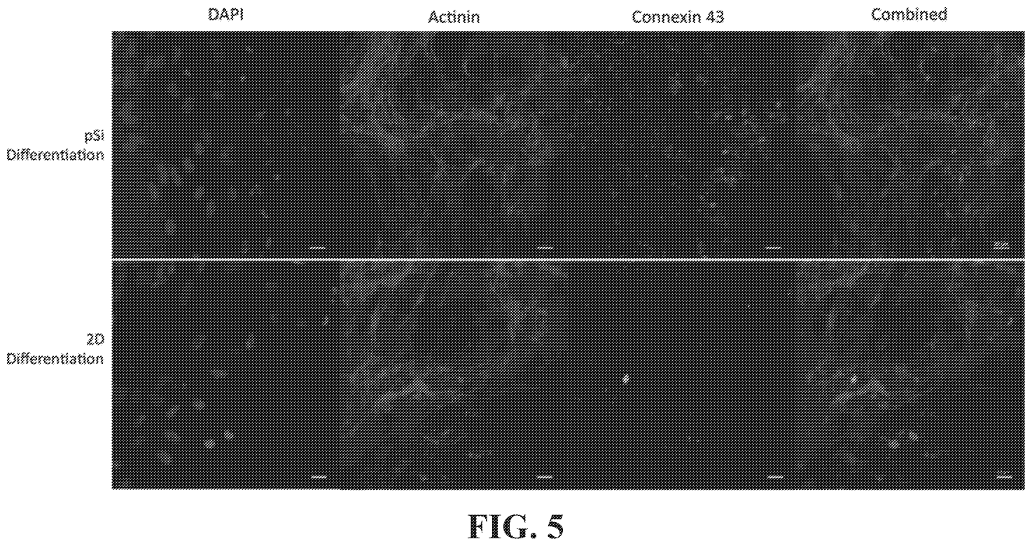

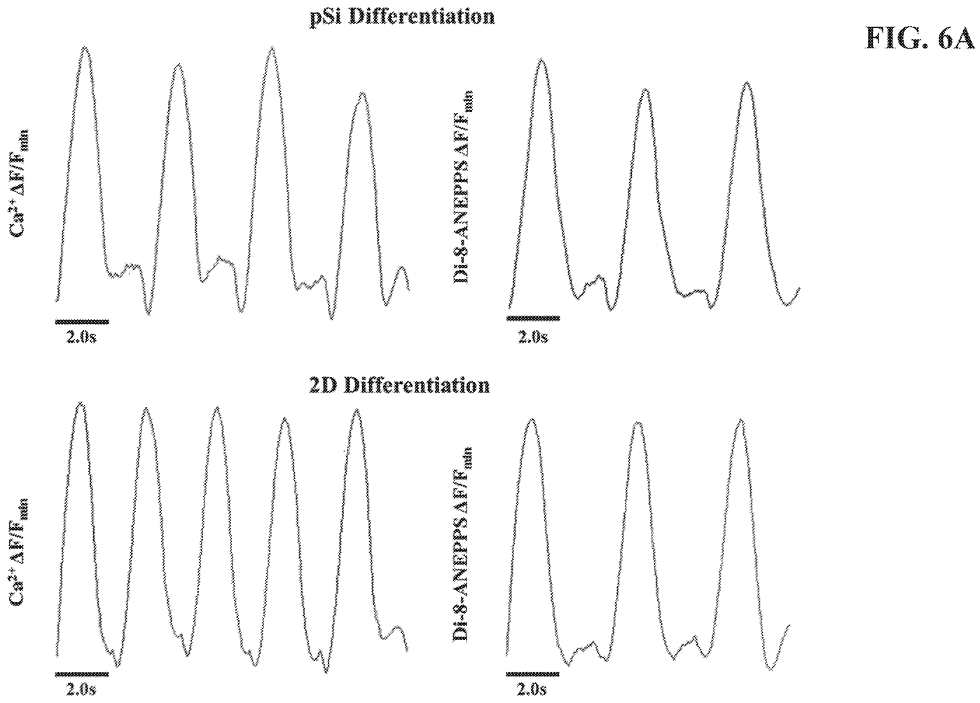

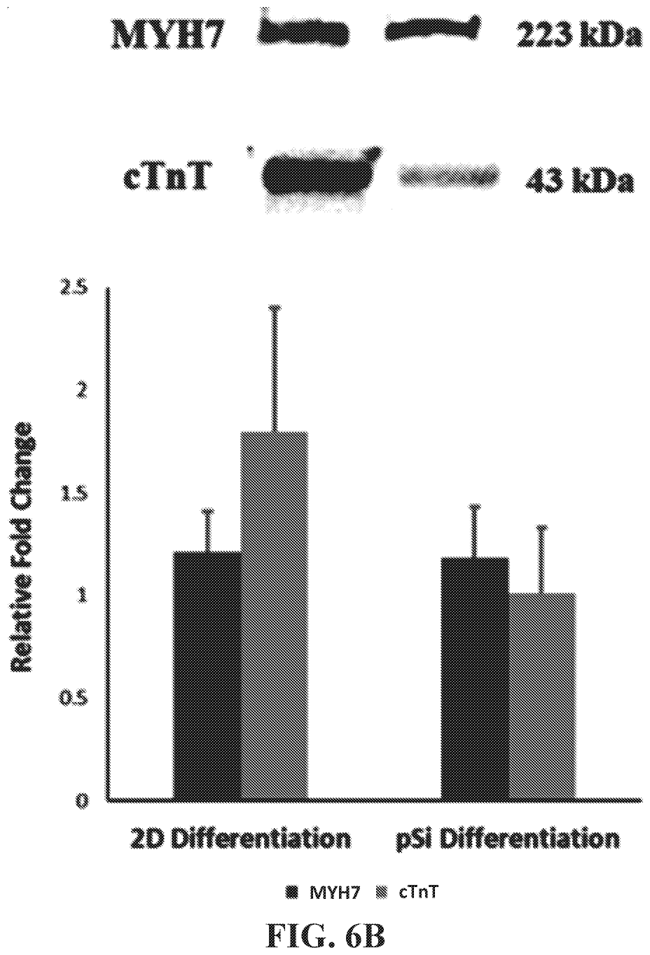

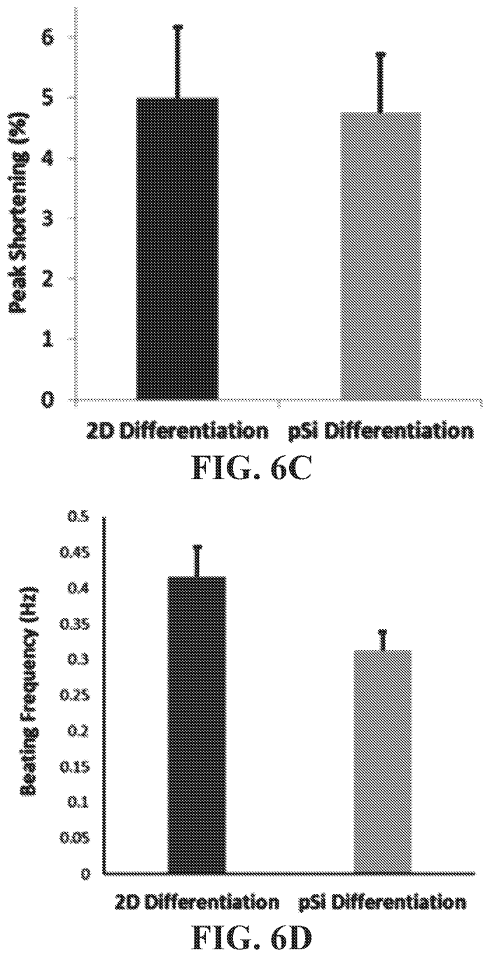

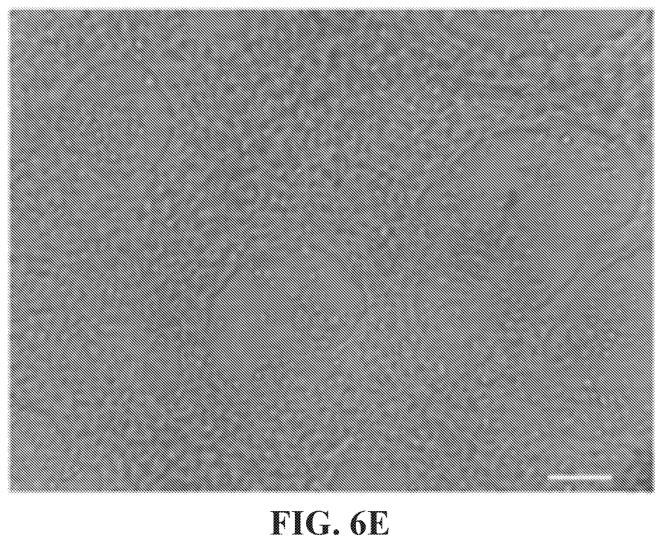

Confocal immunofluorescence staining showed expression of late stage cardiac markers β-MHC and connexin 43 in pSi differentiation at day 30 (FIG. 5). Western blot analysis supports the translation of upregulated late stage cardiac proteins in the pSi differentiation group comparable to 2D differentiation group (FIG. 6B).

### Example 6

Electrophysiological activity and peak shortening of pSi differentiated cells Both the control and experimental groups had spontaneously beating cells at day 18-21. Calcium handling measurements showed pSi differentiated cells spontaneously beat at a frequency of 0.31±0.03 Hz (FIG. 6A). Differentiation with dissolved inhibitors resulted in cells with a beating frequency of 0.48±0.05 Hz (FIG. 6A). Contractile cell peak length shortening was not significantly different between dissolved inhibitor and pSi differentiation groups at 5.0±1.2% and 4.8±1.0%, respectively.

These examples demonstrate that GSK3/Wnt inhibitors released from pSi can be used to differentiate iPSC into cardiac cells, and that controlling the release of GSK3/Wnt inhibitors by pSi encapsulation could produce spontaneously contracting cardiac cells, minimizing handling interactions with the differentiating cells in culture. iPSC did not exhibit cellular apoptosis or necrosis when exposed to pSi. Previous studies have shown that internalization of pSi can induce programmed cell death, and thus the preservation of iPSC cellular activity is likely due to minimal particle internalization. Both pSi formulations were internalized in iPSC but did not show a significant difference in apoptosis and necrosis compared to controls.

The various features and processes described above may be used independently of one another or may be combined in various ways. All possible combinations and subcombinations are intended to fall within the scope of this disclosure. In addition, certain method or process blocks may be omitted in some implementations. The methods and processes described herein are also not limited to any particular sequence, and the blocks or states relating thereto can be performed in other sequences that are appropriate. For example, described blocks or states may be performed in an order other than that specifically disclosed, or multiple blocks or states may be combined in a single block or state. The example blocks or states may be performed in serial, in parallel, or in some other manner. Blocks or states may be added to or removed from the disclosed example embodiments. The example systems and components described herein may be configured differently than described. For example, elements may be added to, removed from, or rearranged compared to the disclosed example embodiments.

Conditional language used herein, such as, among others, “can,” “could,” “might,” “may,” “e.g.,” and the like, unless specifically stated otherwise, or otherwise understood within the context as used, is generally intended to convey that certain embodiments include, while other embodiments do not include, certain features, elements, and/or steps. Thus, such conditional language is not generally intended to imply that features, elements and/or steps are in any way required for one or more embodiments or that one or more embodiments necessarily include logic for deciding, with or without author input or prompting, whether these features, elements and/or steps are included or are to be performed in any particular embodiment. The terms “comprising,” “including,” “having,” and the like are synonymous and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list.

While certain example embodiments have been described, these embodiments have been presented by way of example only and are not intended to limit the scope of the inventions disclosed herein. Thus, nothing in the foregoing description is intended to imply that any particular feature, characteristic, step, module, or block is necessary or indispensable. Indeed, the novel methods and systems described herein may be embodied in a variety of other forms; furthermore, various omissions, substitutions and changes in the form of the methods and systems described herein may be made without departing from the spirit of the inventions disclosed herein. The accompanying claims and their equivalents are intended to cover such forms or modifications as would fall within the scope and spirit of certain of the inventions disclosed herein.

1. A method for inducing cardiomyocyte differentiation in induced pluripotent stem cells (iPS cells), comprising contacting iPS cells in in vitro cell culture media with a porous silica nanoparticle comprising a compound selected from the group consisting of a glycogen synthase kinase 3 (GSK3) inhibitor, a Wnt signaling inhibitor, and a combination thereof, to induce differentiation of cardiomyocytes.

2. The method of claim 1, wherein the Wnt signaling inhibitor is porcupine inhibitor IWP2.

3. The method of claim 1, wherein the GSK-3 inhibitor is CHIR99021.

4. The method of claim 1, wherein the porous silica nanoparticle comprises both the Wnt signaling inhibitor IWP2 and the GSK-3 inhibitor CHIR99021.

5. The method of claim 1, wherein the contacting step comprises simultaneously contacting the iPS cells with a porous silica nanoparticle comprising the Wnt signaling inhibitor IWP2, and a porous silica nanoparticle comprising the GSK-3 inhibitor CHIR99021.

6. The method of claim 5, wherein the porous silica nanoparticle is coated with a biodegradable polymer to achieve delayed-release kinetics of the compound from the nanoparticle.

7. The method of claim 6, wherein the polymer is poly(dl-lactide-co-glycolide) (PLGA), photodegradable poly(ethylene glycol) (PEG), photodegradable poly(caprolactone) (PCL), or photodegradable poly(L-lactide) (PLLA).

8. The method of claim 7, wherein the cell culture media is a defined medium.

9. The method of claim 7, wherein the cell culture media is a serum free medium.

10. The method of claim 7, wherein the porous silica nanoparticles have an average particle size range between 2 and 20 micrometers in diameter.

11. The method of claim 10, wherein the porous silica nanoparticles have a particle shape comprising spheres or rod-shaped particles.

12. The method of claim 11, wherein the porous silica nanoparticles have an average zeta potential between −35 and −40.

13. The method of claim 7, wherein the contacting is conducted in a bioreactor.

14. The method of claim 13, wherein the bioreactor comprises:
a vessel formed of a flexible or rigid disposable container coupled to a means for mixing liquid contents in the container;
at least one media introduction port in fluid contact with the container;
at least one media removal port in fluid contact with the container.

15. The method of claim 14, wherein the bioreactor further comprises a retention screen formed by pores in and opening through the container, wherein the pores are sized to retain the porous silica nanoparticles in the container while fluid cell culture media is removed from the vessel.

16. The method of claim 15, wherein the method further comprises injecting a fluid through the at least one introduction port, and removing fluid cell culture media through the at least one media removal port from the vessel to maintain a substantially steady-state equilibrium of fluid volume within the container throughout the differentiation of cardiomyocytes.

17. The method of claim 16, further comprising transplantation of the induced cardiomyocytes into a mammal in need of such treatment.

18. A porous silica nanoparticle comprising a compound selected from the group consisting of a glycogen synthase kinase 3 (GSK3) inhibitor, a Wnt signaling inhibitor, and a combination thereof.

19. The porous silica nanoparticle of claim 18, wherein the silica nanoparticle is coated with a biodegradable polymer to achieve delayed-release kinetics of the compound from the nanoparticle.

20. The porous silica nanoparticle of claim 19, wherein the polymer is poly(dl-lactide-co-glycolide) (PLGA) photodegradable poly(ethylene glycol) (PEG), photodegradable poly(caprolactone) (PCL), or photodegradable poly(L-lactide) PLLA, wherein the porous silica nanoparticle has a particle shape comprising spheres or rod shaped particles a particle size in a range between 2 and 20 micrometers in diameter and a zeta potential between −35 and −40.

21-23. (canceled)
